(19) United States
(12) Patent Application Publication
(10) Pub. No.: US 2004/0137062 A1
(43) Pub. Date: Jul. 15, 2004

(54) Chronotherapy tablet and methods related thereto

(76) Inventor: Sham Chopra, Brampton (CA)

Correspondence Address: Patrick H. Higgins, Mathews, Collins, Shepherd & McKay, Suite 306, 100 Thanet Circle, Princeton, NJ, 08540, US

(21) Appl. No.: 10/697,473

(22) Filed: Oct. 30, 2003

Related U.S. Application Data
(63) Continuation-in-part of application No. 10/430,142, filed on May 6, 2003.
Application No. 10/430,142 is a continuation-in-part of application No. 10/085,234, filed on Feb. 28, 2002.
(60) Provisional application No. 60/293,701, filed on May 25, 2001.

(51) Int. Cl. A61K 9/24

(52) U.S. Cl. 424/472

(57) ABSTRACT

A chronotherapy tablet is provided for oral administration and the amelioration of at least one chronobiological condition within 24 hours comprising a substantially oblong core having a longitudinal axis, a first end and a second end, the core being comprised of at least two superposed layers of different compositions wherein an interface between each layer is substantially perpendicular to the longitudinal axis of the core and wherein at least one of the layers is a pharmacological composition; a coating which envelops the core, except for at least one exposed release face of the core at at least one end of the core. Methods are provided for the prevention and/or treatment of asthma, arthritis (including, but not limited to, osteoarthritis and rheumatoid arthritis), gastrointestinal disorders, cardiovascular disease (including, but not limited to, hypertension, angina, myocardial infarction, and stroke), and cancer.

Priority is claimed under Title 35 of the United States Code from U.S. application Ser. No. 10/430,142, filed May 6, 2003, which is a continuation-in-part of co-pending U.S. application Ser. No. 10/085,234, filed Feb. 28, 2002. The entire disclosures of each are incorporated herein by reference.

### FIELD OF THE INVENTION

The current invention is drawn toward drug delivery devices constructed to effect proper timing of drug delivery relative to the time of oral administration and thereby significantly increasing the efficiency and efficacy of orally administered agents to ameliorate chronobiological conditions.

### BACKGROUND OF THE INVENTION

Chronobiological patterns are commonly observed with many diseases such as asthma, arthritis (e.g., osteoarthritis and rheumatoid arthritis, for example), gastrointestinal disorders, cardiovascular disease (e.g., hypertension, angina, myocardial infarction, and stroke), and cancer. Coordinating biological rhythms with medical treatment is called chronotherapy.

In recent years chronotherapy has started to play an increasing role in the management of several diseases.

Dosage forms that provide rapid release of drug after a built-in initial time delay have come to be known as pulsed release dosage forms. A variety of types and methods to manufacture such dosage forms have been described in the literature. A dosage form comprising a two part capsule containing a water-swellable material to separate the capsule parts on swelling, for example, has been described in UK2230441. This type of dosage form is extremely expensive and difficult to manufacture. A tablet is described for oral administration which is composed of two layers, one of which contains a drug for immediate release and the other contains a drug for sustained release. In this type of system, drug from not only the immediate release layer but also from the sustained release layer begins to leach out from the start when the dosage form comes in contact with the dissolution medium. Thus, the peak blood concentration is higher than required, increasing the risk of toxic effects. No more than one pulse can be delivered with this system. Other disclosures, for example, EU384514, EU546593 and US 30082230, describe remarkably similar tablets within systems differing only in compositions and methods of manufacture. The described subject matter is taught to accomplish several objectives: 1) a sustained dose for specified time followed by a rapid dose of active ingredient, 2) immediate release of first active ingredient followed by pulse release of a second active ingredient, 3) a delayed release of an active ingredient, or 4) sustained release of a first active ingredient and delayed sustained release of a second active ingredient. EU546593 refers to a stratified tablet. The innermost core is required to be covered on all sides by the next core and so forth. Each layer requires a separate compression step which makes the manufacturing process very laborious and expensive. The compression process also disrupts the inner layers and thus significantly compromises the functionality of the layer dosage form. Successive coatings of drug formulations, moreover, affect the size of the tablet to the extent that the tablet is impractical to administer orally. However, none of the systems can provide immediate release of active ingredient followed by constant release of active ingredient. All provide declining drug release profiles from their sustained release portion of the tablet. U.S. Pat. No. 5,011,692 (1991) relates to a device for subdermal implantation that is capable of releasing active ingredients in a pulse like manner.

Currently, there are no oral dosage forms that can reside in the body for periods longer than 24 hours and deliver required amount of drug at precise time during the circadian rhythms. In absence of such systems, dosage forms have been designed that can be taken a number of hours before the anticipated peak disease time. Uniphyl, a bronchdilator by Purdue Fredrick, is an example of such a dosage form. Taken once a day at dinner time, the drug blood levels reach peak in the morning to improve lung function during the difficult morning hours. Writing in the Apr. 15, 1996 issue of Hospital Practice, Richard Martin, M.D., who directs the division of pulmonary medicine at the National Jewish Center for Immunology and Respiratory Medicine in Denver, stated his belief that “the key to managing [asthma] cases is chronotherapy. I have found that unless treatment improves nighttime asthma, it is hard to improve its daytime manifestations.” For people with severe asthma who wake up several times a night gasping for breath, a good night's sleep can be a dream come true.

It is accordingly considered desirable by those in the field of chronotherapy to provide for a dosage form that will release active drug or drugs in required number of pulses and may also have sustained release portion/s releasing drug at a desired constant rate to match the disease activity.

### SUMMARY OF TH INVENTION

A chronotherapy tablet (2) is provided for oral administration and the amelioration of at least one chronobiological condition within 24 hours comprising a substantially oblong core (4) having a longitudinal axis (6), a first end (8) and a second end (10), the core (4) being comprised of at least two superposed layers (12) of different compositions wherein an interface (14) between each layer is substantially perpendicular to the longitudinal axis (6) of the core (4) and wherein at least one of the layers (12) is a pharmacologically active composition; a coating (16) which envelops the core (4), except for at least one exposed release face (18) of the core (4) at at least one end (20) of the core. Methods are provided for the prevention and/or treatment of asthma, arthritis (including but not limited to osteoarthritis and rheumatoid arthritis), gastrointestinal disorders, cardiovascular disease (including but not limited to hypertension, angina, myocardial infarction, and stroke), and cancer.

### BRIEF DESCRIPTION OF THE FIGURES

FIG. 1 displays an example chronotherapy tablet.

FIG. 2 displays an alternate chronotherapy tablet embodiment.

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

Oral administration refers to oral administration of the chronotherapy tablet, per se.

Stages of drug delivery referred herein correspond to and are accomplished by dissolution of the different layers of the chronotherapy tablets of the present invention under physiological conditions.

A chronotherapy tablet of the present invention is configured structurally to deliver a specific dosage of at least one drug at at least one predetermined period of time relative to the time of oral administration. Chronotherapy tablets of the present invention are specifically constructed for oral administration particularly to release at least one drug in at least one coordinated pulse, particularly for treating diseases which manifest chronobiological conditions such as asthma, arthritis (osteoarthritis and rheumatoid arthritis, for example), gastrointestinal disorders, cardiovascular disease (e.g., hypertension, angina, myocardial infarction, and stroke), and cancer. Embodiments of the present invention, for example, provide a plurality of pulses of pharmacological compositions (including combinations) within 24 hours.

Particularly, drug delivery devices of the current invention are constructed to effect proper timing of drug delivery relative to the time of oral administration and significantly increasing the efficiency and efficacy of the orally administered compounds to ameliorate chronobiological conditions.

Patients are more likely to follow schedules for taking their medications when those medications are formulated as chronotherapies because of better medical results and fewer adverse side effects. The disease can be better contained, which means fewer doctor visits and potential trips to the hospital because of acute flare-ups.

Drugs by means of the present invention are formulated and packaged and orally administered to be released in a variety of different stages and combinations thereof. A first stage of “delivery” begins, for example, immediately after oral administration of a chronotherapy tablet of the present invention. At least one pharmacological compound may immediately begin to be released. This immediate release stage may effect a rapid and short pulse, for example, a fifteen minute burst of drug delivery, or may effect a relatively long period of a substantially constant level of drug delivery. However, different chronotherapy embodiments of the present invention may not begin, for example, to release pharmacological compositions for a significant period of time after oral administration. Embodiments of the present invention even delay drug delivery for hours after oral administration of the chronotherapy tablet. Preferred embodiment of chronotherapy tablets described herein intended for administration at bedtime, for example, are constructed to delay drug delivery for a variable number of hours to provide therapy when needed. Accordingly, the first stage of “delivery” may in fact be a delay stage lasting from about 5 to about 8 hours.

A second stage of delivery, and so forth, e.g., third, fourth, fifth, and the like, similar to the first stage described supra, and depending upon the nature of the first stage and chronobiological condition to be treated, may effect a rapid and short pulse, for example, a fifteen minute burst of drug delivery, or may effect a relatively long period of a substantially constant level of drug delivery, or may effect a period of delay by means of a “delay layer” further described infra. Oral administration of chronotherapy tablets of the present invention accomplishes drug delivery in as many stages as may be necessary or practical over, for example, a 24 hour period of time.

Particularly, chronotherapy tablets of the present invention are able to, for example, (a) deliver one or two drugs three times a day at desired time intervals; (b) bypass the stomach and release drug in the intestine in two pulses (each one at a determined interval); (c) release a pharmacological composition instantly followed by a constant rate release for a desired period, followed by instant drug release, followed by a release at a constant rate for desired period, followed by instant release; and (d) deliver many different combinations of pulse release, delay, and constant release.

Chronotherapy tablets of the present invention are structurally configured dosage forms that fundamentally enable drug delivery to be synchronized with biological rhythms of disease activity. Embodiments of the invention are configured to deliver a predetermined dosage of drug at at least one certain predetermined period of time relative to the initial oral administration of the therapeutic entity. Embodiments of the invention also enable the delivery of at least one drug over a plurality of different periods of time. Chronotherapy tablets of the present invention also encompass embodiments wherein a plurality of different drugs are each delivered at a predetermined dosage at a plurality of periods of time relative to the time of oral administration. The therapeutic entity of the invention is capable of providing several pulses of drug delivery; three pulses, for example, of the same drug, different drugs, or the same or different combinations, in 24 hours. Pulses may be delayed as described herein; however, between pulses one or more drugs for example, can be released at a constant rate. Therapeutic entities described herein are structurally configured to effect various combinations of pulse release, constant release, and delayed release of compounds upon oral administration.

Preferred Embodiments

A preferred embodiment of the present invention is a chronotherapy tablet comprised of a substantially oblong core comprising a first end and a second end. Oblong, as used herein refers to oblong or oblong and tapered at the end(s), elliptical, or cylindrical. Although oblong cores are preferred, other shaped cores having a longitudinal axis are feasible as well, including but not limited to square-ended, rectangular, pentagonal, hexagonal, septagonal, and octagonal. The core is comprised of at least two layers of different compositions wherein the interface between each layer is substantially perpendicular to the longitudinal axis of the core. The term “composition(s)”, by itself, is used herein to refer to both delay compositions (inactive materials) without drug(s) and pharmacological compositions comprising one or more drugs for delivery. At least one of the layers is a pharmacologically active composition. The chronotherapy tablet further comprises a coating which completely covers the core except for at least one exposed release face of a first layer at at least one end of the core. “Release face” is used herein to refer to an exposed portion of a layer of the core composition. The initial release face, before administration of the chronotherapy tablet, may be any shape, including three dimensional, e.g., semi oblong, particularly in view of the definition of oblong presented herein. Any exposed portion of the release face, however, that initially extends beyond the cover, dissolves in a relatively short period of time after oral administration, e.g., fifteen minutes, to yield a release face that is substantially perpendicular to the longitudinal axis of the core during dissolution of the core.

Accordingly, a chronotherapy tablet of the present invention is comprised of a substantially oblong core having a longitudinal axis, a first end and a second end, the core being comprised of at least two superposed layers of different compositions wherein an interface between each layer is substantially perpendicular to the longitudinal axis of the core and wherein at least one of the layers is a pharmacologically active composition; a coating which envelops the core; except for at least one exposed release face of the core at at least one end of the core.

Embodiments of the present invention may have one release face at a first end of the core -or- two release faces, one release face at a first end of the core and one release face at a second end of the core. The core may be symmetrical or asymmetrical with reference to the superposed layers of the compositions of the core.

Preferred embodiments of the present invention are chronotherapy tablets described herein wherein at least one of the layers of compositions is a delay layer. Example embodiments of this type are chronotherapy tablets wherein a first exposed layer (by means of a release face) is a delay layer. In double release face embodiments of the current invention both primarily exposed layers at each end of the core (the first end and the second end) may therefore initially be release faces of delay layers. Delay layers are in many different embodiments positioned within the “stacked” of core of different pharmacologically active compositions to provide for proper timing of drug release to meet chronobiological needs.

Chronotherapy tablets of the present invention may comprise, for example, at least three layers of pharmaceutical compositions and two delay layers wherein the three layers of pharmaceutical compositions (not necessarily different) are each separated from each other by a delay layer.

Arthritis

Nonsteroidal anti-inflammatory agents that may be used for the treatment of arthritis are acetic acid derivatives including indole derivatives, for example, sodium or potassium diclofenac, etodolac, indomethacin, ketorolac tromethamine, sulindac and tolmetin sodium; cyclooxygenase-2 inhibitors, for example, celecoxib and rofecoxib; fenamates, for example, mefenamic acid and floctafenine; oxicams, for example, meloxicam, piroxicam, piroxicam cyclodextrin, tenoxicam; propionic acid derivatives like fenoprofen, flurbiprofen, ibuprofen, ketoprofen, naproxen, naproxen sodium oxaprozin and tiaprofenic acid and salicylic acid derivatives like acetylsalicylic acid and diflunisal.

Chronobiological patterns have been observed with arthritis pain. People with osteoarthritis, the most common form of the disease, tend to have less pain in the morning and more at night. But for people with rheumatoid arthritis, the pain usually peaks in the morning and decreases as the day wears on. Recent animal studies showing that joint inflammation in rats fluctuates over a 24-hour period support these observations by both patients and physicians.

Chronotherapy for all forms of arthritis uses standard treatment, nonsteroidal anti-inflammatory drugs and corticosteroids; however, the dosages are timed to ensure that the highest blood levels of the drug coincide with peak pain.

Osteoarthritis tends to exhibit most symptoms in the evening and at night; whereas, symptoms of rheumatoid arthritis, however, peaks in the morning and decreases as the day wears on. Rheumatoid arthritis is a chronic inflammatory autoimmune disorder. The cardinal signs of rheumatoid arthritis are stiffness, swelling and pain of one or more joints of the body, characteristically most severe in the morning. Rheumatoid arthritis shows a marked circadian variation in its symptoms. Typically, within the population of rheumatoid arthritis sufferers, the severity of joint pain, swelling and stiffness is generally about 3 times higher between about 7AM to about 11AM when compared to normal bedtime. Hand strength is also generally lower by as much as 30% in these morning hours. Common treatment for arthritis, for example, are corticosteroids and nonsteroidal anti-inflammatory drugs. The usual dose of naproxen, a nonsteroidal anti-inflammatory drug, for example, is 500 mg per day in divided doses. The dosage may be increased to 1000 mg per day depending upon the patient's response. Increased dosages amplifies the risk of adverse effects such as peptic ulcers, which has demonstrated fatalities, particularly in elderly.

Chronotherapy tablets of the present invention will ensure that the peak blood level of naproxen, for example, is produced and maintained, with a single minimal tablet (375 mg total, for example) in the needed hours of the morning to coincide with peak symptoms, i.e., pain and stiffness. Chronotherapy tablets of the present invention moreover minimize toxic effects of the drug.

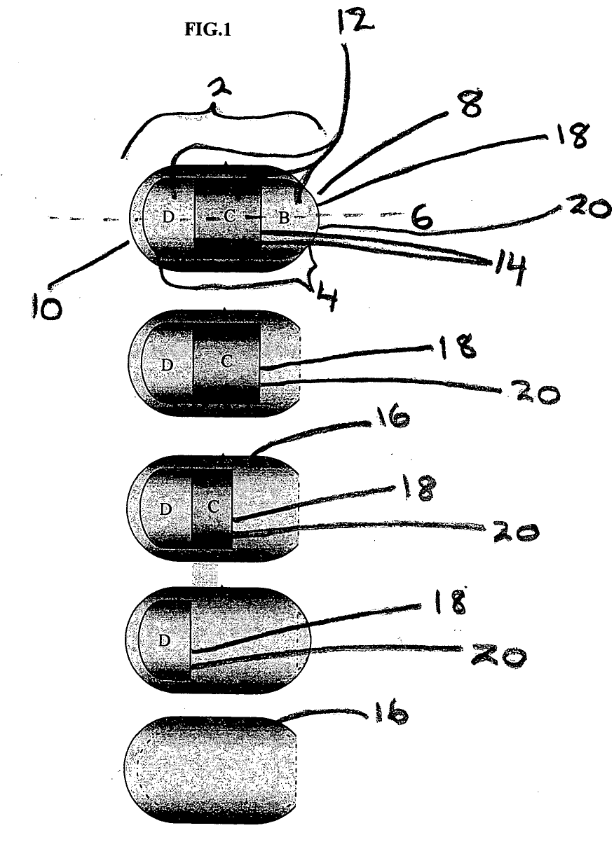

Preferred chronotherapy tablets of the present invention, for example, may comprise a second layer adjacent to a first exposed delay (release face) layer which second layer comprises a therapeutically effective amount of a drug selected from the group consisting essentially of (corticosteroids and nonsteroidal anti-inflammatory drugs), a third layer adjacent to the second layer which third layer comprises a therapeutically effective amount of a drug selected from the group consisting essentially of (corticosteroids and nonsteroidal anti-inflammatory drugs); and, wherein formulation of the delay layer provides for substantially complete dissolution of the delay layer between about 5-9 hours after oral administration or dissolution of the tablet under physiological conditions. Embodiments of the present invention are, for example, with reference to FIG. 1, wherein the second layer comprises a therapeutically effective amount of an NSAID, e.g., ibuprofen and/or naproxen, and the third layer comprises a therapeutically effective amount of an NSAID, e.g., ibuprofen and/or naproxen. A chronotherapy tablet of the present invention is preferred, for example, wherein the second layer, with reference to FIG. 1, comprises between about 175 mg-675 mg naproxen wherein (after oral administration) said naproxen is substantially completely releasable within about 15 minutes of substantially complete dissolution of the delay layer under physiological conditions; and, the third layer comprises between about 100 mg-250 mg naproxen that is releasable, after substantially complete dissolution of the second layer, at a substantially constant rate over a period of about five hours under physiological conditions. A particularly preferred species embodiment of this type is a chronotherapy tablet wherein the second layer comprises about 250 mg naproxen and the third layer comprises about 125 mg naproxen.

Cardiovascular

Compounds that may be used for antihypertensive and antanginal activity are alpha-adrenergic blocking agent: doxazosine mesylate, prazosin hydrochloride and terazosine hydrochloride dehydrate; Alpha & Beta-adrenergic blocking agent: labetalol hydrochloride; Beta-adrenergic, blocking agents, selective non ISA: atenolol, bisoprolol, esmolol hydrochloride and metoprolol hydrate; Beta adrenergic blocking agent, non selective ISA: oxprinolol hydrochloride and pindolol; Beta-adrenergic blocking agents, non-selective, non-ISA: nadolol, propanolol hydrochloride and timolol maleate; Centrally acting antiadrenergic agents: clonidine hydrochloride and methyldopa; Calcium Channel Blockers: amlodipine besylate, diltiazem hydrochloride; felodipine, nifedipine and verapamil Hydrochloride; Vasolidators: diazoxide, epoprostenol sodium, hydralazine hydrochloride, minoxidil and nitroglycerine; Potassium sparing agents: amiloride hydrochloride, spironolactone and triamterene; Angiotensin converting enzyme inhibitors: benazipril hydrochloride, captopril, enalapril maleate, lisinopril, and ramipril; Angiotensin II receptor antagonists: valsartan, candesartan cilexitil and losartan potassium

Blood pressure (BP) is sensitive to circadian variations and follows a predictable cycle, i.e., a substantial rise upon early-morning awakening, a plateau during daily activities, and a decline of approximately 20% during sleep. Morning surges in BP pose significant risks to patients with hypertension yet more cases are treated with traditional therapies that do little to specifically address these potentially dangerous peaks. Studies have identified a 49% higher risk of stroke, a 40% higher risk of myocardial infraction and a 29% higher risk of cardiac death, particularly in patients with hypertension, particularly between about 6AM and about 12PM.

Embodiments of the chronotherapy tablet of the present invention deliver antihypertensive, antianginal compounds, for example, diltiazem, at various times and amounts to match the disease conditions throughout the day.

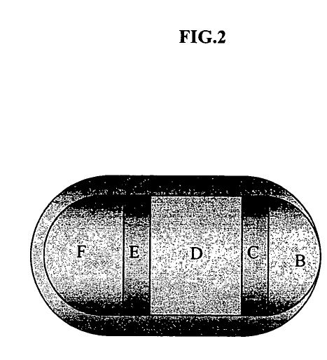

A chronotherapy tablet with reference to FIG. 2, for example, may comprise at least three layers of pharmaceutical compositions and two delay layers wherein the three layers of pharmaceutical compositions, not necessarily different, are each separated from each other by a delay layer. Accordingly, a preferred chronotherapy tablet, with reference to FIG. 2, is constructed wherein the first layer comprises a therapeutically effective amount of a drug selected from the Calcium Channel Blocker group consisting essentially of nifedipine or diltiazem hydrochloride or felodipine or verapamil hydrochloried, further comprising

a second layer adjacent to the first layer which second layer is a delay layer containing placebo and it delays the release of second dose by eight hours,

a third layer adjacent to the second layer which third layer comprises a therapeutically effective amount of a drug selected from the Calcium Channel Blocker group consisting essentially of nifedipine or diltiazem hydrochloride or felodipine or verapamil hydrochloried,

a fourth layer adjacent to the third layer which fourth layer is a delay layer containing placebo and it delays the release of third dose by eight hours,

a fifth layer adjacent to the fourth layer which fifth layer comprises a therapeutically effective amount of a drug selected from the Calcium Channel blocker group consisting essentially of nifedipine or diltiazem hydrochloride or felodipine or verapamil hydrochloried; and,

wherein formulation of the second and fourth delay layers each provide for substantially complete dissolution of the delay layer to require between about 5-9 hours, each, during dissolution of the core under physiological conditions. An embodiment of this type is a chronotherapy tablet wherein the first layer comprises a therapeutically effective amount of diltiazem, the third layer comprises a therapeutically effective amount of diltiazem, and the fifth layer comprises a therapeutically effective amount of diltiazem. A preferred embodiment is wherein the first layer comprises between about 25 mg-100 mg diltiazem wherein said diltiazem within the first layer is substantially completely releasable within about 15 minutes of oral administration of the tablet or dissolution of the tablet under physiological conditions, and the third layer comprises between about 50 mg-150 mg diltiazem wherein said diltiazem within the third layer is substantially completely releasable within about 15 minutes of substantially complete dissolution of the second layer; and,

the fifth layer comprises between about 80 mg-200 mg diltiazem wherein said diltiazem within the fifth layer is substantially completely releasable within about 15 minutes of substantially complete dissolution of the fourth layer. A particularly preferred chronotherapy tablet of this type is wherein the first layer comprises about 60 mg diltiazem, the third layer comprises about 180 mg diltiazem, and the fifth layer comprises about 120 mg diltiazem.

Asthma

Asthma is a common breathing problem affecting nearly five percent of Americans. Asthma is a disease of the lung airways (bronchi). Narrowing of the openings of the airways (caused by spasm, swelling of the lining, and/or mucus accumulation) can lead to shortness of breath, wheezing, or coughing. Causes of asthma include allergies, cold air, air pollutants, drugs, cigarette smoke, molds, exercise, and infections. Asthma attacks (rapid worsening of symptoms) typically occur in episodes. Intervals between attacks can be days, weeks or years. With severe asthma, attacks can occur daily.

Scientists now believe that asthma attacks vary according to the time of day. The occurrence of asthma attacks is not random. Asthma symptoms are frequently worse at night (nocturnal) for a majority of asthma sufferers.

Asthma is often at its worst in the early AM, for example, when cortisol levels are at their lowest. Normal lung function undergoes circadian changes and reaches a low point in the early morning hours. This dip is particularly pronounced in people with asthma. The incidence of asthma attacks was recently demonstrated to be more than 100 times greater during nighttime, especially around 4AM, than it was during the middle of the day. Accordingly, preferred embodiments of the present invention are accordingly employed to relieve asthma symptoms during early morning hours.

Drugs that may be used for asthma therapy include, but are not limited to, Alpha and Beta-adrenergic Agonists: epinephrine bitartrate, epinephrine hydrochloride, racemic; Beta-2-adrenergic agonists/Selective: fenoterol hydrobromide, formoterol fumarate, salbutamol, salbutamol sulphate and terbutaline sulphate; Systemic Xanthines: aminophylline, oxtriphyllin and theophylline.

Chronotherapy for asthma is aimed at getting maximal effect from bronchodilator medications during the early morning hours. A chronotherapy tablet with reference to FIG. 1, for example, may comprise at least two layers of pharmaceutical compositions and one delay layer wherein the two layers of pharmaceutical compositions, not necessarily different, are separated from each other by the delay layer. Accordingly, a preferred chronotherapy tablet, with reference to FIG. 1, is constructed wherein the first layer comprises a therapeutically effective amount of a drug selected from the beta-2-adrenergic Agonists group consisting essentially of salbutamol sulphate, terbutaline sulfate and the Systemic Xanthines group consisting essentially of aminophyllin and theophylline; the tablet further comprising,

a second layer adjacent to the first layer which second layer is a delay layer, for example, containing placebo which delays the release of a third layer (second dose) by about five (5) to nine (9) hours, preferably about eight (8) hours; the tablet further comprising,

a third layer adjacent to the second layer which third layer comprises a therapeutically effective amount of a drug selected from a beta-2-adrenergic agonists group consisting essentially of salbutamol sulphate and terbutaline sulfate, or a Systemic Xanthines group consisting essentially of aminophyllin and theophylline wherein formulation of the second delay layers provides for substantially complete dissolution of the delay layer to require between about 5-9 hours, each, during dissolution of the core under physiological conditions. An embodiment of this type is a chronotherapy tablet, for example, wherein the first layer comprises a therapeutically effective amount of salbutamol and the third layer comprises a therapeutically effective amount of salbutamol. A preferred embodiment is wherein the first layer comprises between about 2 mg to about 4 mg salbutamol sulphate wherein said salbutamol within the first layer is substantially completely releasable within about 15 minutes of oral administration of the tablet or dissolution of the tablet under physiological conditions, and the third layer comprises between about 2 mg to about 4 mg salbutamol wherein said salbutamol within the third layer is substantially completely releasable within about 15 minutes of substantially complete dissolution of the second layer.

Materials

Materials to be used in forming delay layers, e.g., layer B in FIG. 1, and Layers C and E in FIG. 2 are not limited to any particular species. Preferred materials are therapeutically inactive and solubilize, dissolve, degrade, or erode relatively slowly under physiological conditions.

The delay layer(s) are preferably substantially impenetrable to solvent or enclosed drug compounds within an adjacent layer until the delay layer has substantially completely dissolved. The delay layer(s) may be comprised of well known diluent/s, dissolution rate modifier/s, lubricant/s, polymer/s, surfactant/s and/or platicizer/s commonly used in the art of pharmacy and formulations related thereto.

Layer D in FIG. 1, for example, for slow release of a therapeutic compound, may be composed of the same or similar materials to Layer B in the same figure. Similarly, Layers C and E in FIG. 2 may also contain one or more drug compounds.

In a high shear blender/granulator of the Fielder type or Diosna type, the drug is blended with a slow dissolving polymer which comprises the shaped core. If required, a soluble diluent may also be added. The blend is subsequently granulated with water or an organic solvent or a mixture of water and an organic solvent. Alternatively a slow dissolving or colloidal dispersion forming polymer may be dissolved or dispersed in a solvent and added to the blend while mixing continuously. The mixture thus granulated is then dried at a suitable temperature and milled through a screen with an appropriate opening. The granules prepared are then mixed with a soluble or insoluble lubricant.

Layers C in FIG. 1 and Layers B, D and F in FIG. 2, for example, in addition to at least one therapeutic compound, may comprise well known therapeutically inactive diluent/s, and/or binder/s, and/or disintegrant/s, and/or lubricant/s, and/or dissolution rate modifier/s, and/or polymer/s, and/or surfactant/s commonly used in the art of pharmacy and formulations related thereto.

The coat layer comprises an insoluble or slowly soluble polymer and, optionally, a soluble component which leaches out of the coat rendering it porous, weak and susceptible to disintegration shortly after release of the active ingredient is complete. Layer A, the coat layer in FIG. 1 and FIG. 2 is composed of soluble and/or insoluble polymers, pore formers and lubricants. The coat layer may contain diluents and/or plasticizers.

Examples of suitable pore-forming materials that may be used in the present invention include but are not limited to, alkali and alkaline earth metal salts, organic compounds such as polysaccharides, organic aliphatic alcohols including diols, polyols, polyhydric alcohol, polyalkylene glycol, polyglycol, and the like. The pore-forming elements preferably have a size within about 0.1 to about 200 microns. In a presently preferred embodiment, the coat comprises 1 to 50% of pore former based on the weight of the polymer. Preferred pore-forming materials include sugars, e.g., dextrose, fructose, glucose, dextrates, sorbitol and propylene glycol, and carbowax.

Certain chronotherapy tablet embodiments of the present invention are comprised of a substantially oblong core, otherwise as defined herein, and a coating which envelops the core comprising water-soluble pore-forming material(s) that substantially leach out of the coat and thereby introduce mechanical instability and allow the coat to disintegrate after release of the active compound is complete. The pores, however, due to the timing of their formation/completion (about 8 hours to about 22 hours after ingestion), do not substantially affect the release rate of the active ingredient. In other words, in contrast to the prior art, in these certain embodiments, the instant invention effects drug release at the exposed face(s) not through the coat. Particularly, certain embodiments of the present invention are designed to form pores after the drug release period is substantially complete. The purpose of the coat is to prevent the coated surface of the core from imbibing dissolving fluids. The drug in the core dissolves from the uncoated faces when exposed to the dissolving fluid. The coat remains intact throughout the delivery period but disintegrates prior to evacuation from the colon. Whereas upon oral administration the chronotherapy tablet effects the amelioration of at least one chronobiological condition within 24 hours.

Examples of dissolution rate modifiers that either alone or in combination may be used in the present invention as polymers either as soluble polymers or polymers that produce clear colloidal dispersion in water, include but are not limited to hydroxypropyl cellulose, hydroxyethyl cellulose, hydroxypropyl methylcellulose; polyvinylpyrrolidone, methyl cellulose, soluble modified starches, gelatin, acacia, polyethyleneoxide, and polyethyleneglycol. Water insoluble polymers that may be used in the present invention, include but are not limited to cellulose acetate, cellulose acetate butyrate, polyvinyl alcohol, ethyl cellulose, methacrylic acid copolymers, insoluble modified starches, and polypropylene oxide. Biodegradable polymers that may be used in the present invention include but are not limited to polyglycolide, poly-L-lactide, poly-D,L-lactide, caprolactone, polyamino acids, polyorthoesters and polyanhydrides. One skilled in the art will recognize other polymers with similar properties which also may be used and the invention is not limited to the specific polymers listed herein.

Examples of suitable soluble diluents for use in the present invention that may be incorporated into the core, include, but are not limited to, lactose, sucrose, carbowax, dextrates, glucose, fructose, soluble starch, sorbitol, mannitol, hydroxypropyl cellulose, hydroxyethyl cellulose, hydroxypropyl methylcellulose, polyvinylpyrrolidone, methyl cellulose, soluble modified starches, gelatin, acacia.

Examples of pH sensitive diluents include cellulose acetate phthalate, hydroxypropyl methylcellulose phthalate, polyvinylacetate phthalate and polymethacrylate. Examples of suitable insoluble diluents that may be used include, but are not limited to, calcium sulfate, dicalcium phosphate, microcrystalline cellulose, insoluble modified starches and starch.

Examples of suitable lubricants that may be used include, but are not limited to, stearic acid, sodium stearate, calcium stearate, magnesium stearate, sodium stearyl fumarate and sodium lauryl sulfate.

Drug compounds, pharmaceutical agents and or otherwise therapeutic entities for use in chronotherapy tablets of the present invention include, but are not limited to, cardiovascular drugs, stroke treatment agents, respiratory therapies, analgesics, anti-arthritic agents, gastrointestinal products, muscle-relaxants, muscle-contractants, anti-inflammatory agents, hormonal agents, and diuretics.

Examples of drugs that can be formulated into the chronotherapy tablets of the present invention include acetic acid derivatives including indole derivatives, for example, sodium or potassium diclofenac, etodolac, indomethacin, ketorolac tromethamine, sulindac and tolmetin sodium; cyclooxygenase-2 inhibitors for example celecoxib and rofecoxib; fenamates for example mefenamic acid and floctafenine; oxicams for example meloxicam, piroxicam, piroxicam cyclodextrin, tenoxicam; propionic acid derivatives like fenoprofen, flurbiprofen, ibuprofen, ketoprofen, naproxen, naproxen sodium oxaprozin and tiaprofenic acid and salicylic acid derivatives like acetylsalicylic acid and diflunisal; Alpha-adrenergic blocking agents including doxazosine mesylate, prazosin hydrochloride and terazosine hydrochloride dehydrate; Alpha & Beta-adrenergic blocking agents including labetalol hydrochloride; Beta-adrenergic, blocking agents, selective non ISA: atenolol, bisoprolol, esmolol hydrochloride and metoprolol hydrate; Beta adrenergic blocking agent, non selective ISA: oxprinolol hydrochloride and pindolol; Beta-adrenergic blocking agents, non-selective, non-ISA: nadolol, propanolol hydrochloride and timolol maleate; Centrally acting antiadrenergic agents: clonidine hydrochloride and methyldopa; Calcium Channel Blockers: amlodipine besylate, diltiazem hydrochloride; felodipine, nifedipine and verapamil Hydrochloride; Vasolidators: diazoxide, epoprostenol sodium, hydralazine hydrochloride, minoxidil and nitroglycerine; Potassium sparing agents: amiloride hydrochloride, spironolactone and triamterene; Angiotensin converting enzyme inhibitors: benazipril hydrochloride, captopril, enalapril maleate, lisinopril, and ramipril; Angiotensin II receptor antagonists: valsartan, candesartan cilexitil and losartan potassium Beta-adrenergic Agonists: epinephrine bitartrate, epinephrine hydrochloride, racemic; Beta-2-adrenergic agonists/Selective: fenoterol hydrobromide, formoterol fumarate, salbutamol, salbutamol sulphate and terbutaline sulphate; Systemic Xanthines: aminophylline, oxtriphyllin and theophylline.

Manufacture of chronotherapy tablets of the present invention comprises the following basic steps.

Granulation

Layers for similar purpose or function can be prepared employing similar granulation compositions and methods described herein and/or known in the art. Delay composition layers, for example, may consist of standard tabletting excipients including binders, diluents, dissolution modifiers and lubricants. Therapeutically active composition layers generally contain at least one pharmacologically active agent (drug), excipients, diluents, disintegrants, binders and/or lubricants. Sustained release layers, particularly for hydrophilic drugs, for example, require the presence of dissolution regulators to prevent rapid and immediate dissolution of the entire core. Chronotherapy tablets of the present invention enable the release rate to be optimized for all types of drugs. Granulations may be prepared using well known wet or dry methods. All ingredients, including diluents and dissolution rate modifiers, are blended together in a dry method in an appropriate size blender, for example, to prepare a delay layer composition. To prepare pharmacologically active composition layers, drugs are included.

The preparation of rapid release pharmacologically active composition layers involves the blending together of all ingredients including pharmacologically active agents, diluents and disintegrants. The blend may be milled and sieved, if desired, through a sieve with an appropriate size mesh, the mesh size is chosen according to the application. The blend is then mixed with an appropriate lubricant.

Delay layer compositions may be formulated in wet granulation by blending diluents with slow dissolving polymer/s. Pharmacologically active composition layers may similarly be formulated with active compound. The blend is subsequently granulated with water or an organic solvent or a mixture of water and an organic solvent.

Alternatively, a slow dissolving or dispersing polymer may be dissolved or dispersed in a solvent and added to the blend while mixing continuously. The mixture thus granulated is dried at a suitable temperature and milled through a screen with an appropriate opening. The granules prepared are then mixed with a soluble or insoluble lubricant.

Compression of Granulations

Prepared granulations are compressed into multi-layered cores on a conventional multi-layer press using appropriately shaped tooling. With the currently available equipment, like Korsch TRP 900-5S available from Korsch America Inc. in the USA, the number of core layers is limited to five. The number of core layers, however, can be increased with the availability of appropriate equipment in the future.

Compression Coating of the Multi-Layer Core

Pre-compressed bodies are compression coated using a core-coater fitted with a specially designed tooling for placing bodies precisely in the dies. Approximately fifty percent of the required coating material is transferred to the die and the core is placed precisely at the desired location on the top of the granules in the die. The remaining fifty-percent of the coating granules are then placed on top of the core. The punches are then brought closer together to compress all the components of the device. An example of a suitable core-coater is manufactured by Korsche Pressing. Any machine can be used that allows the geometrically shaped core to be compression coated, preferably a machine that uses a device to pick up the bodies for the precise placement in the center of the die. The surface of the multi-layer core, excluding one or both faces parallel to the tablet layers is then coated using a compression-coating machine, Core-Coater available from Korsch America Inc. in the USA, with a specially designed transfer mechanism. The coating material used to encase the active core is prepared by mixing a blend of soluble and insoluble polymers with appropriate plasticizers. The blend thus prepared is lubricated with lubricant/s. All ingredients used in this technology are pharmaceutically acceptable. There are consequently no toxicological issues with the technologies.

Although specific methods for manufacturing the chronotherapy tablets are described herein, numerous modifications and alternative process steps will be apparent to those skilled in the art. Accordingly, this description is to be construed as illustrative only and is for the purpose of teaching those skilled in the art methods to prepare the invention. These processes may be varied substantially without departing from the spirit of the invention.

### EXAMPLES

### Example I

See, FIG. 1. Layer B is a delay layer composition which takes approximately eight hours to dissolve. Layer C contains about 250 mg of naproxen releasable within fifteen minutes after the dissolution of Layer B. Layer D contains about 125 mg naproxen releasable at a constant rate of about 25 mg/hr over a period of about five hours. The coat A disintegrates after dissolution of Layer C is complete. The coat, layer A, comprises an insoluble or slowly soluble polymer and, optionally, a soluble component which leaches out of the coat rendering it porous, weak and susceptible to disintegration shortly after release of the active ingredient is complete.

### Example II

See, FIG. 2. Layer C and Layer E are delay composition layers which require approximately eight hours after oral administration to dissolve (under physiological conditions). Layer B contains about 60 mg diltiazem. Layer D contains about 180 mg diltiazem and Layer F contains about 120 mg of diltiazem. Each of these layers, B, D, and F, once exposed, require about fifteen to about twenty five minutes to release its drug contents under physiological conditions. Layer B disintegrates and releases all its drug within fifteen minutes of administration. Coat, A, comprises an insoluble or slowly soluble polymer and, optionally, a soluble component which leaches out of the coat rendering it porous, weak and susceptible to disintegration shortly after release of the active ingredient is complete.

### Example III

Manufacture of a Naprosyn Chronotherapy Tablet

Granulation for a Delay Layer Composition

In an appropriate blender, lactose mono hydrate is blended with a low viscosity hydroxypropyl cellulose which comprise the dissolution-based core. Other suitable polymers and diluents are known in the art and examples are described supra. The blend is granulated with water and the granulation thus prepared is dried. The blend is milled and sieved through a 20 mesh sieve. The blend is then mixed with sodium stearate.

Granulation for Modified Release Naprosyn Layer

Milled naprosyn is blended with hydroxypropyl cellulose. The blend is granulated with water in a high shear granulator and dried at 50 degree centigrade. The granulation is milled through a 20 mesh screen. The milled granules are lubricated with sodium stearate.

Granulation for Immediate Release Naprosyn Layer

Milled naprosyn, lactose spray dried, avicel and calcium carmellose are blended and milled through 20 mesh screen. The granules are then lubricated with magnesium stearate.

Compression of Granules into Triple Layer Tablet

Modified release core granules are transferred to the tablet die of a triple layer compression machine and pre-compressed lightly followed by the transfer of immediate release granules and light pre-compression and then followed by inactive modified release core granules. The lightly compressed triple layer granules are finally compressed to the appropriate hardness. Examples of suitable triple layer core compressing machines that may be used are known in the art and supplied by, for example, Elizabeth Hata, Killian & Co. Inc. (Pennsylvania, USA), Thomas Engineering (Illinois, USA) and Fette America Inc. (New Jersey, USA).

Coating of the Naprosyn Triple Layer Core

An insoluble polymer, ethyl cellulse type N7 and powdered sugar, a pore former, are blended in a suitable blender with magnesium stearte, an insoluble lubricant. The blend may be sieved through a screen with an appropriate size mesh to remove any large size agglomerate from the compression coat granules. An appropriate amount of these granules is fed to the bottom of the die of the Korsch core-coater. The pre-compressed triple layer core with its inactive side in up position is transferred on the top of the bed of the granules in the die using a specially fabricated transfer and positioning device. The coat around the entire core surface, except for the top face, is then applied by compression.

### Example IV

Manufacture of a Diltiazem Chronotherapy Tablet

Granulation for Delay Composition Layers

In an appropriate blender, calcium sulphate is blended with hydroxypropyl cellulose which comprises the dissolution-based core. Suitable polymers and diluents are known in the art and examples are described above. The blend is granulated with water, dried at 50 degrees centigrade, and milled through a 20 mesh sieve. The blend is then mixed with sodium stearate.

Granulation for a Immediate Release Diltiazem Layer

In an appropriate blender, a required amount of dilitiazem and lactose are blended and milled through 20 mesh screen. The blend is granulated with aqueous solution of povidone, dried and milled through a 20-mesh screen. The granules thus prepared are blended with starch and lubricated with magnesium stearate.

Compression of the Granules into a Five Layer Tablet

Appropriate amount of dilitiazem core granules are transferred to the tablet die of a five layer Korsch compression machine and compressed lightly followed by the transfer of delay layer composition granules and light pre-compression followed by dilitiazem composition granules followed by delay composition layer granules and then another dilitiazem composition layer. The lightly compressed five layer granules are finally compressed to the appropriate hardness.

Compression Coating of the Dilitiazem Chronotherapy Core

Ammonio methacrylate copolymer type B is blended with methacrylic acid copolymer type B and sprayed with triethyl citrate. The blend is lubricated with magnesium stearate. The blend may be sieved through a screen with an appropriate size mesh to remove any large size agglomerate from the compression coat granules. An appropriate amount of these granules is fed to the bottom of the die of the Korsch core-coater. The pre-compressed five layer core with its appropriate active side in up position is transferred on the top of the bed of the granules in the die using a specially fabricated transfer and positioning device. The coat around the entire core surface, except for the top face, is then applied by compression.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described compositions and modes for carrying out the invention which are obvious to those skilled in the art or related fields are intended to be within the scope of the following claims.

What is claimed is:

1. A chronotherapy tablet comprising:
a substantially oblong core having a longitudinal axis, a first end and a second end, the core being comprised of at least two superposed layers of different compositions wherein an interface between each layer is substantially perpendicular to the longitudinal axis of the core and wherein at least one of the layers is a pharmacologically active composition;
a coating which envelops the core, except for;
at least one exposed release face of the core at at least one end of the core;
whereas upon oral administration the chronotherapy tablet effects the amelioration of at least one chronobiological condition within 24 hours.

2. A chronotherapy tablet according to claim 1 comprising two layers of pharmaceutical compositions separated by a layer of a different composition.

3. A chronotherapy tablet according to claim 1 comprising at least two layers of different pharmaceutical compositions.

4. A chronotherapy tablet according to claim 2 wherein two layers of different pharmaceutical compositions are separated by a layer of a different composition.

5. A chronotherapy tablet according to claim 3 wherein two layers of the same pharmaceutical compositions are separated by a layer of a different pharmaceutical composition.

6. A chronotherapy tablet according to claim 4 comprising three layers of three different pharmaceutical compositions.

7. A chronotherapy tablet according to claim 2 comprising two layers of the same pharmaceutical composition separated by a layer of a different composition.

8. A chronotherapy tablet according to claim 1 wherein at least one of the layers of compositions is a delay layer.

9. A chronotherapy tablet according to claim 3 wherein the first layer is a delay layer.

10. A chronotherapy tablet according to claim 4 wherein the layer of a different composition is a delay layer.

11. A chronotherapy tablet according to claim 7 wherein the layer of a different composition is a delay layer.

12. A chronotherapy tablet according to claim 2 comprising at least three layers of pharmaceutical compositions and two delay layers wherein the three layers of pharmaceutical compositions are each separated from each other by a delay layer.

13. A chronotherapy tablet according to claim 9 comprising
a second layer adjacent to the first layer which second layer comprises a therapeutically effective amount of a drug, and
a third layer adjacent to the second layer which third layer comprises a therapeutically effective amount of a drug, and
wherein the delay layer provides for substantially complete dissolution of the delay layer between about 5 to about 9 hours after oral administration; and,
the drug is selected from the group consisting essentially of a nonsteroidal anti-inflammatory agent, an acetic acid derivative, acetylsalicylic acid, an indole derivative, sodium or potassium diclofenac, etodolac, indomethacin, ketorolac tromethamine, sulindac, tolmetin sodium, cyclooxygenase-2 inhibitor, celecoxib, rofecoxib, a fenamate, mefenamic acid, floctafenine, an oxicam, meloxicam, piroxicam, piroxicam cyclodextrin, tenoxicam, a propionic acid derivative, fenoprofen, flurbiprofen, ibuprofen, ketoprofen, naproxen, naproxen sodium, oxaprozin, a tiaprofenic acid derivative, a salicylic acid derivative, and diflunisal.

14. A chronotherapy tablet according to claim 13 wherein the second layer comprises a therapeutically effective amount of naproxen and the third layer comprises a therapeutically effective amount of naproxen.

15. A chronotherapy tablet according to claim 14 wherein the second layer comprises between about 175 mg-675 mg naproxen wherein said naproxen is substantially completely releasable within about 15 minutes of substantially complete dissolution of the delay layer under physiological conditions; and,
the third layer comprises between about 100 mg-250 mg naproxen that is releasable, after substantially complete dissolution of the second layer, at a substantially constant rate over a period of about 5 hours under physiological conditions.

16. A chronotherapy tablet according to claim 15 wherein the second layer comprises about 250 mg naproxen and the third layer comprises about 125 mg naproxen.

17. A chronotherapy tablet according to claim 12 wherein the first layer comprises a therapeutically effective amount of a drug, further comprising
a second layer adjacent to the first layer which second layer is a delay layer,
a third layer adjacent to the second layer which third layer comprises a therapeutically effective amount of a drug,
a fourth layer adjacent to the third layer which fourth layer is a delay layer,
a fifth layer adjacent to the fourth layer which fifth layer comprises a therapeutically effective amount of a drug,
wherein the second and fourth delay layers each provide for substantially complete dissolution of the delay layer to require between about 5 to about 9 hours, each, during dissolution of the core under physiological conditions; and,
the drug is selected from the group consisting essentially of an alpha-adrenergic blocking agent, doxazosine mesylate, prazosin hydrochloride, terazosine hydrochloride dehydrate, an Alpha & Beta-adrenergic blocking agent, labetalol hydrochloride, a Beta-adrenergic blocking agent, a selective non ISA, atenolol, bisoprolol, esmolol hydrochloride, metoprolol hydrate, a Beta adrenergic blocking agent, a non selective ISA, oxprinolol hydrochloride, pindolol, a Beta-adrenergic blocking agent, a non-selective non-ISA, nadolol, propanolol hydrochloride, timolol maleate, a centrally acting antiadrenergic agent, clonidine hydrochloride, methyldopa, a Calcium Channel Blocker, amlodipine besylate, diltiazem hydrochloride, felodipine, nifedipine, verapamil Hydrochloride, a Vasolidator, diazoxide, epoprostenol sodium, hydralazine hydrochloride, minoxidil, a Potassium sparing agent, amiloride hydrochloride, spironolactone, triamterene, an Angiotensin converting enzyme inhibitor, benazipril hydrochloride, captopril, enalapril maleate, lisinopril, ramipril, an Angiotensin II receptor antagonists, valsartan, candesartan cilexitil, and losartan potassium.

18. A chronotherapy tablet according to claim 13 wherein the first layer comprises a therapeutically effective amount of diltiazem, the third layer comprises a therapeutically effective amount of diltiazem, and the fifth layer comprises a therapeutically effective amount of diltiazem.

19. A chronotherapy tablet according to claim 18 wherein the first layer comprises between about 25 mg-100 mg diltiazem wherein said diltiazem within the first layer is substantially completely releasable within about 15 to about 25 minutes of oral administration, and
the third layer comprises between about 50 mg-150 mg diltiazem wherein said diltiazem within the third layer is substantially completely releasable within about 15 to about 25 minutes of substantially complete dissolution of the second layer; and,
the fifth layer comprises between about 80 mg-200 mg diltiazem wherein said diltiazem within the fifth layer is substantially completely releasable within about 15 to about 25 minutes of substantially complete dissolution of the fourth layer.

20. A chronotherapy tablet according to claim 19 wherein the first layer comprises about 60 mg diltiazem, the third layer comprises about 180 mg diltiazem, and the fifth layer comprises about 120 mg diltiazem.

21. A chronotherapy tablet according to claim 2 wherein a first layer comprises a therapeutically effective amount of a drug,
a second layer adjacent to the first layer is a delay layer,
a third layer adjacent to the second layer comprises a therapeutically effective amount of a drug, and
wherein a therapeutically effective amount of a drug in the first layer is substantially completely releasable within about 15 minutes of oral administration of the tablet, and
the second layer provides for substantially complete dissolution of the layer to require between about 5 to about 9 hours during dissolution of the core under physiological conditions, and
a therapeutically effective amount of a drug in the third layer is substantially completely releasable within about 15 minutes of substantially complete dissolution of the second layer; and,
wherein the drug is selected from the group consisting essentially of a beta-2-adrenergic agonist, salbutamol sulphate, terbutaline sulfate, a Systemic Xanthine, aminophyllin, and theophylline.

22. A chronotherapy tablet according to claim 21 wherein the first layer comprises a therapeutically effective amount of salbutamol, and the third layer comprises a therapeutically effective amount of salbutamol.

23. A chronotherapy tablet according to claim 22 wherein the first layer comprises between about 2 mg to about 4 mg salbutamol sulphate, and the third layer comprises between about 2 mg to about 4 mg salbutamol sulphate.

24. A method of treatment and/or prevention of a chronobiological condition comprising orally administering a chronotherapy tablet comprising
a substantially oblong core having a longitudinal axis, a first end and a second end, the core being comprised of at least two superposed layers of different compositions wherein an interface between each layer is substantially perpendicular to the longitudinal axis of the core and wherein at least one of the layers is a pharmacologically active composition;
a coating which envelops the core, except for;
at least one exposed release face of the core at at least one end of the core;
whereas upon oral administration the chronotherapy tablet effects the amelioration of at least one chronobiological condition within 24 hours.

25. A method according to claim 24 of treatment and/or prevention of a chronobiological condition selected from the group consisting essentially of asthma, arthritis, gastrointestinal disorder, cardiovascular disease, and cancer.

26. A method according to claim 25 of treatment and/or prevention of a chronobiological condition of arthritis comprising orally administering a chronotherapy tablet comprising at least two layers of different pharmaceutical compositions and wherein the first layer is a delay layer, a second layer adjacent to the first layer which second layer comprises a therapeutically effective amount of a drug, and
a third layer adjacent to the second layer which third layer comprises a therapeutically effective amount of a drug, and
wherein the delay layer provides for substantially complete dissolution of the delay layer between about 5 to about 9 hours after oral administration; and,
the drug is selected from the group consisting essentially of a nonsteroidal anti-inflammatory agent, an acetic acid derivative, acetylsalicylic acid, an indole derivative, sodium or potassium diclofenac, etodolac, indomethacin, ketorolac tromethamine, sulindac, tolmetin sodium, cyclooxygenase-2 inhibitor, celecoxib, rofecoxib, a fenamate, mefenamic acid, floctafenine, an oxicam, meloxicam, piroxicam, piroxicam cyclodextrin, tenoxicam, a propionic acid derivative, fenoprofen, flurbiprofen, ibuprofen, ketoprofen, naproxen, naproxen sodium, oxaprozin, a tiaprofenic acid derivative, a salicylic acid derivative, and diflunisal.

27. A method according to claim 25 of treatment and/or prevention of a chronobiological condition of cardiovascular disease comprising orally administering a chronotherapy tablet comprising a first layer which comprises a therapeutically effective amount of a drug,
a second layer adjacent to the first layer which second layer is a delay layer,
a third layer adjacent to the second layer which third layer comprises a therapeutically effective amount of a drug,
a fourth layer adjacent to the third layer which fourth layer is a delay layer,
a fifth layer adjacent to the fourth layer which fifth layer comprises a therapeutically effective amount of a drug,
wherein the second and fourth delay layers each provide for substantially complete dissolution of the delay layer to require between about 5 to about 9 hours, each, during dissolution of the core under physiological conditions; and,
the drug is selected from the group consisting essentially of an alpha-adrenergic blocking agent, doxazosine mesylate, prazosin hydrochloride, terazosine hydrochloride dehydrate, an Alpha & Beta-adrenergic blocking agent, labetalol hydrochloride, a Beta-adrenergic blocking agent, a selective non ISA, atenolol, bisoprolol, esmolol hydrochloride, metoprolol hydrate, a Beta adrenergic blocking agent, a non selective ISA, oxprinolol hydrochloride, pindolol, a Beta-adrenergic blocking agent, a non-selective non-ISA, nadolol, propanolol hydrochloride, timolol maleate, a centrally acting antiadrenergic agent, clonidine hydrochloride, methyldopa, a Calcium Channel Blocker, amlodipine besylate, diltiazem hydrochloride, felodipine, nifedipine, verapamil Hydrochloride, a Vasolidator, diazoxide, epoprostenol sodium, hydralazine hydrochloride, minoxidil, nitroglycerine, a Potassium sparing agent, amiloride hydrochloride, spironolactone, triamterene, an Angiotensin converting enzyme inhibitor, benazipril hydrochloride, captopril, enalapril maleate, lisinopril, ramipril, an Angiotensin II receptor antagonists, valsartan, candesartan cilexitil, and losartan potassium.

28. A method according to claim 25 of treatment and/or prevention of a chronobiological condition of asthma comprising orally administering a chronotherapy tablet comprising a first layer which comprises a therapeutically effective amount of a drug,
a second layer adjacent to the first layer which is a delay layer,
a third layer adjacent to the second layer which comprises a therapeutically effective amount of a drug, and
wherein a therapeutically effective amount of a drug in the first layer is substantially completely releasable within about 15 minutes of oral administration of the tablet, and
the second layer provides for substantially complete dissolution of the layer to require between about 5 to about 9 hours during dissolution of the core under physiological conditions, and
a therapeutically effective amount of a drug in the third layer is substantially completely releasable within about 15 minutes of substantially complete dissolution of the second layer; and,
wherein the drug is selected from the group consisting essentially of a beta-2-adrenergic agonist, salbutamol sulphate, terbutaline sulfate, a Systemic Xanthine, aminophyllin, and theophylline.
